(19) United States
(12) Patent Application Publication
(10) Pub. No.: US 2003/0028148 A1
(43) Pub. Date: Feb. 6, 2003

(54) Tubing holder and stabilizer

(75) Inventors: Clifton G. Hampton, Burleson, TX (US); Gary Pedersen, Bedford, TX (US)

Correspondence Address: Charles D. Gunter, Jr., BRACEWELL & PATTERSON, LLP, Suite 1600, 201 Main Street, Fort Worth, TX, 76102, US

(73) Assignee: Welcon, Inc.

(21) Appl. No.: 09/920,536

(22) Filed: Aug. 1, 2001

(51) Int. Cl. A61M 5/32

(52) U.S. Cl. 604/180

(57) ABSTRACT

A disposable medical tubing holder and stabilizer comprising a base layer and a pair of opposing hook and loop regions located on a top planar surface of a base layer. A primary adhesive region selectively sized to receive a length of medical tubing is located between the opposing hook and loop regions on the top planar surface so as to separate the opposing hook and loop regions. The opposing hook and loop regions are foldable about the primary adhesive region to thereby form an engaged sheath about the medical tubing to secure the medical tubing to the tubing holder. A secondary adhesive region is located on the bottom planar surface of the base layer and is engageable on a target surface for stabilizing a medical tubing on the target surface.

### BACKGROUND OF THE INVENTION

1. Technical Field

The present invention relates generally to disposable medical devices and, particularly, to a method and apparatus for temporarily fastening and stabilizing medical tubing.

2. Description of the Related Art

In an intravenous infusion (IV) system for plasma, glucose water or other therapeutic agents, a cannula or catheter is injected into a vein or artery of the patient and is coupled by an extended flexible plastic tubing to a fluid source. In extracting fluid from a patient, a similar tubing is extended between a medical device inserted in the patient through a nostril or other body cavity and a fluid receiver. In all such situations, it is vital that the tubing adjacent to its point of coupling to the catheter or other medical device be immobilized in some way. In the absence of an immobilizer, a substantial tug on the tubing, which may occur inadvertently or when the patient changes his position, will displace or dislodge the catheter and may cause the catheter to cut the vein wall or to pull out of the vein, thereby causing the infusion to infiltrate the region surrounding the vein and result in a loss of intravenous therapy. This mishap necessitates restarting infusion therapy which is both inconvenient and possibly painful to the patient. In order to effect immobilization of the tubing, the usual hospital practice is to improvise an immobilizer by means of strips of conventional adhesive tape. These strips are placed over the tubing and adhered to the skin of the patient at a position adjacent the point of infusion or insertion.

It is also important that the immobilizer used to stabilize the tubing not depend on the ability of the tape to adhere to the skin of the patient. Modern pressure-sensitive tapes, when applied to a skin surface, afford strong adherence. This is particularly true where the entire underface of the tape engages a corresponding skin surface. When typical adhesive tape is applied over a flexible tubing, however the region of adhesive contact between the tubing and the tape is limited, allowing the bonding to be disrupted. This disruption can cause the tubing to slide and increase the risk of displacement or dislodgement of the catheter. Adherence of the tape to the patient's skin can also cause injury in the case of geriatric patients or other patients with fragile or thin skin.

Previous attempts to address this problem have several drawbacks. Many of the proposed devices are not disposable. Others do not secure the medical tubing properly and can allow the tubing to slide. Still other devices clamp onto the flexible tubing and may occlude the fluid passage. What is needed is a method and apparatus to economically fasten and stabilize medical tubing which reduces strain and provides a positive strain relief.

### BRIEF SUMMARY OF THE INVENTION

It is an object of the present invention to provide a tubing holder and stabilizer that provides a positive strain relief.

It is another object of the present invention to provide a tubing holder and stabilizer that can be simply and easily installed.

It is yet another object of the present invention to provide a tubing holder and stabilizer that is relatively inexpensive and disposable.

The above objects are achieved by providing a disposable medical tubing holder and stabilizer which is formed from a base layer having a pair of opposing hook and loop regions located on a top planar surface of the base layer. A primary adhesive region, selectively sized to receive a length of medical tubing, is located between the opposing hook and loop regions on the top planar surface and separates the opposing hook and loop regions. The opposing hook and loop regions are foldable about the primary adhesive region to thereby form an engaged sheath about the medical tubing to secure the medical tubing to the tubing holder. A secondary adhesive region is located on a bottom planar surface of the base layer directly below one of the opposing hook and loop regions. The secondary adhesive region is engageable on a target surface for stabilizing the medical tubing on the target surface. The target surface can be a patient's skin or a stand-off material such as a gauze wrap or strain reliefboard. The adhesive regions are initially covered with peel-off protective coverings.

To use the tubing holder, the user first removes the peel-off protective coating to expose the primary adhesive region. A section of the tubing is secured to the primary adhesive region and the base layer is folded so the hook and loop regions mate and form an engaged sheath about the engaged tubing. Once the tubing is securely attached to the tubing holder, the secondary adhesive region is exposed by removing the peel-off covering. The secondary adhesive region is then attached to a strain relief surface located a convenient distance from the ultimate body entry site. The attachment site should be such that the medical patient does not feel any discomfort at the body entry site as a result of the tubing being too tight or too loose, thereby putting unnecessary pressure on the body entry site. The tubing holder provides excellent strain relief properties because the primary adhesive region forms an engaged sheath about the tubing and secures the tubing to the tubing holder while the secondary adhesive region secures the tubing holder to a strain relief surface thereby preventing undesired movement of the tubing. The tubing holder is simple in design and inexpensive to manufacture and represents an improvement in the existing art in terms of simplicity of design and materials.

Additional objects, features and advantages will be apparent in the written description which follows.

### BRIEF DESCRIPTION OF THE DRAWINGS

The novel features believed characteristic of the invention are set forth in the appended claims. The invention itself however, as well as a preferred mode of use, will best be understood by reference to the following detailed description of an illustrative embodiment when read in conjunction with the accompanying drawings, wherein:

FIG. 1 is a perspective view of the disposable medical tubing holder and stabilizer of the invention in use on the arm of a medical patient.

FIG. 2 is an isolated view of the tubing holder of the invention showing the tubing holder being applied to an IV line.

FIG. 3 is a top view of the disposable medical tubing holder and stabilizer of the invention.

FIG. 4 is a side view of the disposable medical tubing holder and stabilizer of the invention.

FIG. 5 is a side view similar to FIG. 4 but showing medical tubing being positioned on the primary adhesive surface of the device

FIG. 6 is a side view of the device similar to FIG. 5 but showing the opposing hook and loop regions of the device being engaged.

### DETAILED DESCRIPTION OF THE PREFERRED EMBODIMENT

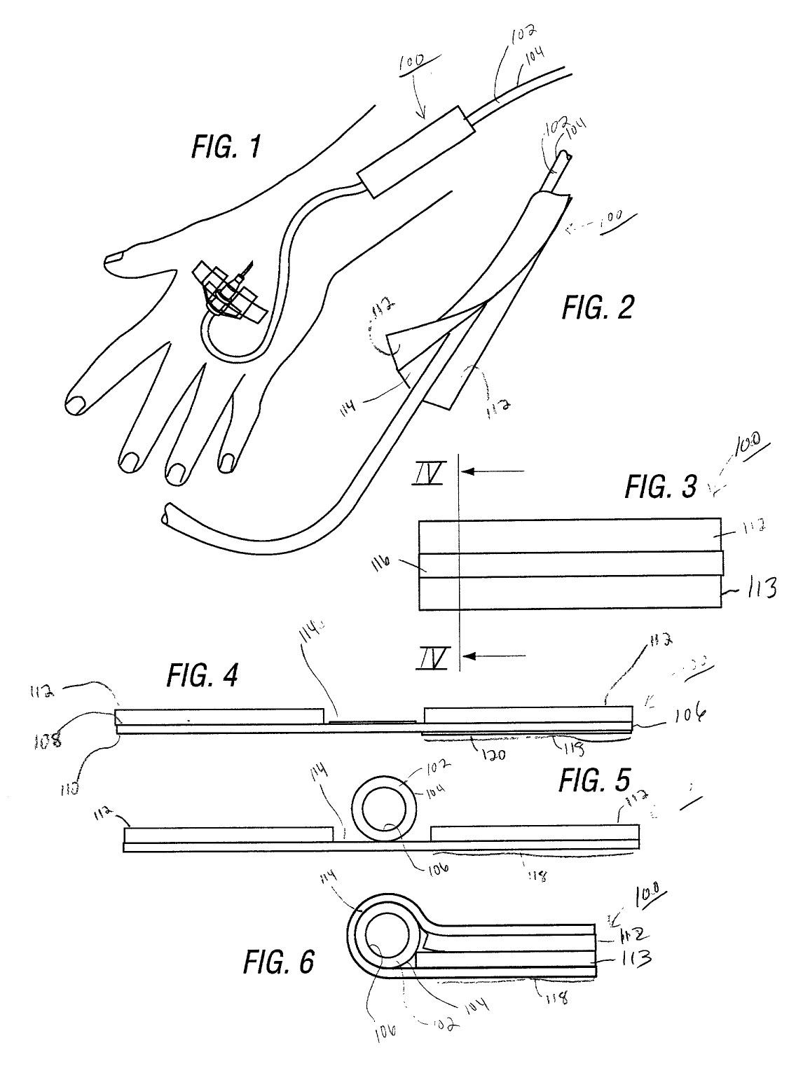

FIG. 1 of the drawings show a disposable medical tubing holder and stabilizer, the device being designated generally as 100. The tubing holder 100 is used to hold and secure medical tubing 102. Tubing 102 can be any type of relatively small diameter tubing or cords such as the tubing generally found in medical hospitals i.e. intravenous tubing, vacuum tubes, nasal gastric tubes, cautery cords, electrical cords, and the like. The tubing may be formed from a variety of commercially available materials such as rigid PVC, flexible PVC, low durometer PVC, polyethylene, polypropylene, TPE's and TPR's, high impact styrene, polycarbonate, polyurethane, hytrel, kraton, noryl, other types of acrylic, nylon, plastic, vinyl, or rubber. The outside diameter 104 of the tubing 102 used with the device of the invention is typically less than approximately 0.5 inches and is generally less than 0.25 inches. One advantage of the device of the invention is the ease of adaptability of the device to secure a range of diameter of medical tubing.

The tubing holder 100 includes abase layer 106 (FIG. 4). The base layer 106 maybe a sheet of disposable plastic material, such as a polyolefin sheet, a porous foam plastic material such as foamed polyurethane, dense cotton, or any other similar material. A variety of commercially available materials can be used for the base layer 106 so long as the base layer is flexible, light in weight, and inexpensive to manufacture. The thickness of the base layer 106 is not critical but is generally relatively thin, approximately less than 10 mm, so as not to affect flexibility or weight. The shape of the base layer 106 is generally rectangular having a length which exceeds its width. The base layer 106 has a top planar surface 108 and a bottom planar surface 110.

A pair of engageable hook and loop regions 112, 113 preferably formed of commercially available velcro®, are located on the top planer surface 108 and run along opposing sides of the base layer 106 parallel to the length thereof. The length of each hook and loop region 112, 113 is approximately the same length as the base layer 106. However, there could be several separate and distinct hook and loop regions 112, 113 spaced along the length of the base layer 106 with a predetermined distance between each region. The hook and loop regions 112, 113 offer the ability to easily adjust, reposition, or replace the tubing 102.

The width of each region 112, 113 will be determined based upon the overall width of the base layer 106, the outside diameter 104 of the tubing 102, and the desired strength of the bond formed when the hook and loop regions 112 are brought togther. The strength of the bond produced by the hook and loop regions 112, 113 will obviously be determined based upon the amount of contact surface area of the hook and loop regions. In the example device illustrated in FIG. 3, the width of the hook and loop regions 112 is about 0.25 inches.

A primary adhesive region 114 (FIG. 4) is located between the opposing hook and loop regions 112, 113 on the top planar surface 108. The primary adhesive region 114 may be covered by a peel-of protective covering such as a peel-away silicone treated paper 116 (FIG. 3) that covers and protects the primary adhesive region 114. In use, the paper 116 is removed to expose the primary adhesive region 114. The length of the primary adhesive region 114 is preferably the same length as the base layer 106 and runs the entire length of the base layer 106 so as to separate the opposing hook and loop regions 112. However, a plurality of the primary adhesive regions 114 could be spaced along the length of the base layer. The length and width of the primary adhesive region 114 are selected to accommodate the particular size of the tubing 102 to be stabilized.

Thus, the width of the primary adhesive region 114 is determined by the overall width of the base layer 106 in addition to the outside diameter 104 of the tubing 102. If the outside diameter 104 of the tubing 102 is relatively large, then the width of the primary adhesive region 114 should also be relatively large. In the example device shown in FIGS. 3-6, the primary adhesive region 114 has a width which is approximately the same as the width of each of the opposing hook and loop regions 112.

As shown in FIG. 6, the primary adhesive region 114 forms an engaged sheath around the tubing 102 when the opposing hook and loop regions 112 are brought together. The purpose of the primary adhesive region 114 is to fixedly secure the tubing 102 to the tubing holder 100 and provide a positive strain relief. Also, the primary adhesive region 114 provides the ability to secure multiple tubing 102 of various sizes at the same time. The adhesive should be sufficiently strong to withstand pulling on the tubing 102, but not so strong as to prevent subsequent separation when it is desirable to remove the tubing holder 100 from the tubing 102 for repositioning or replacement of the tubing 102.

A secondary adhesive region 118 is located on the bottom planer surface 110 of the base layer 106. The secondary adhesive region 118 may be covered by a peel-away silicone treated paper 120 (FIG. 5) that covers and protects the secondary adhesive region 118. In use, the paper 120 is removed to expose the secondary adhesive region 118. The secondary adhesive region 118 runs along the bottom surface 110 and is preferably the same length as the base layer 106. However, there could be several secondary adhesive regions 118 spaced along the length of the base layer 106 with a predetermined distance between each region 118.

The width of the secondary adhesive region 118 is not critical and depends on the overall width of the base layer 118. Generally, the secondary adhesive region 118 is located directly below one of the hook and loop regions 112 and may be the same width as the hook and loop region 112. Also, the secondary adhesive region 118 could be located directly below the primary adhesive region 114 or any other location on the bottom surface 110 that would allow the secondary adhesive region 118 to perform the function of securing the tubing holder 100 to a target surface.

The adhesive on the secondary adhesive region 118 must be strong enough to ensure the tubing holder 100 can securely engaged on the target surface. The target surface may be an exposed area on a medical appliance, a strain relief board, a medical patient's exposed skin, a medical patient's skin that has been prewrapped in common gauze wrap and finished with a wrap of medical tape, or any other target surface that will provide the appropriate strain relief. Often the patient's skin is not the best strain relief surface, especially on those that it most often used such as pediatric and geriatric patients because the thin skin can become damaged during strain relief or during removal.

To use the tubing holder 100, the user first removes the peel-away silicone treated paper 116 to expose the primary adhesive region 114. A predetermined section of the tubing 102 is secured to the primary adhesive region 114 and the base layer 106 is folded so the hook and loop regions 112 mate and form an engaged sheath about the tubing 102. Once the tubing 102 is securely attached to the tubing holder 100, the peel-away silicone treated paper 120 is removed to expose the secondary adhesive region 118. The secondary adhesive region 118 then is attached to a strain relief surface a predetermined distance from the body entry site. The predetermined distance should be such that the medical patient does not feel any discomfort at the body entry site as a result of the tubing 102 being too tight or too loose and thereby put unnecessary pressure on the body entry site.

An invention has been provided with several advantages. The present tubing holder 100 provides excellent strain relief properties because the primary adhesive region forms an engaged sheath about the tubing and secures the tubing to the tubing holder while the secondary adhesive region secures the tubing holder to a strain relief surface thereby preventing movement of the tubing from reaching the body entry site. The hook and loop regions 112, 113 offer the ability to adjust and or replace the tubing without disturbing the secondary strain relief surface attachment. The primary adhesive region 114 provides the ability to secure multiple tubing 102 of various sizes at the same time. The tubing holder is inexpensive in design and manufacture and provides a truly disposable tubing holder due to the simplicity of design and materials.

It should be understood that various alternatives to the embodiments of the invention described herein may be employed in practicing the invention. For example the tubing holder may be used to secure the wires inside electrical components or the cords of electrical devices. It is intended that the following claims define the scope of the invention and that structures and methods within the scope of these claims and their equivalents be covered thereby.

What is claimed is:

1. A disposable medical tubing holder and stabilizer comprising:
a base layer having a top planar surface and a bottom planar surface;
a pair of opposing hook and loop regions located on the top planar surface of the base layer, the opposing hook and loop regions each having a length and a width;
a primary adhesive region located between the opposing hook and loop regions on the top planar surface and running substantially the entire length thereof so as to separate the opposing hook and loop regions, the primary adhesive region being selectively sized to receive a length of tubing to be stabilized;
a secondary adhesive region located on the bottom planar surface of the base layer directly below one of the opposing hook and loop regions; and
wherein the secondary adhesive region is engageable on a target surface for stabilizing a medical tubing on the target surface, and wherein the opposing hook and loop regions are foldable about the primary adhesive region when a medical tubing is located on the primary adhesive region to thereby form an engaged sheath about the medical tubing to stabilize the medical tubing on the target surface.

2. The tubing holder and stabilizer of claim 1, wherein the target substrate is a medical patient's exposed skin area.

3. The tubing holder and stabilizer of claim 1, wherein the target substrate is an exposed surface area on a medical appliance.

4. The tubing holder and stabilizer of claim 1, wherein the base layer is generally rectangular in shape having a length which exceeds a width thereof, and wherein the opposing hook and loop regions run along opposing sides of the base layer parallel to the length thereof.

5. The tubing holder and stabilizer of claim 4, wherein the primary adhesive layer which separates the opposing hook and loop regions has a width which is approximately the same as the width of each of the opposing hook and loop regions.

6. The tubing holder and stabilizer of claim 5, wherein the secondary adhesive layer runs along a selected side edge of the bottom planar surface of the base layer for substantially the entire length thereof.

7. The tubing holder of claim 6, wherein the base layer is made of a disposable plastic material.

8. The tubing holder of claim 1, wherein the primary and secondary adhesive layers are initially covered with a peel-off protective covering.

9. A method for holding and stabilizing medical tubing comprising the steps of:
providing a tubing holder comprising:
a base layer having a top planar surface and a bottom planar surface, the top planar surface having opposing hook and loop regions separated by a primary adhesive region; and
a secondary adhesive region located on the bottom planar surface of the base layer;
placing a portion of a medical tubing to be stabilized on the primary adhesive region;
folding the base layer so that the hook and loop regions mate and form an engaged sheath about the medical tubing with the medical tubing contacting the primary adhesive region;
securing the second adhesive region to a strain relief surface, thereby holding and stabilizing the medical tubing.

10. The method of claim 9, wherein the strain relief surface is a common board.

11. The method of claim 9, wherein the strain relief surface is a wrap of medical tape.

12. The method of claim 9, wherein the strain relief surface is an exposed area of a patient's skin.

13. The method of claim 9, wherein the primary and secondary adhesive regions are provided with a peel-off protective covering.
